(19) United States
(12) Patent Application Publication
(10) Pub. No.: US 2012/0101259 A1
(43) Pub. Date: Apr. 26, 2012

(54) Counter-current extraction of oil seed protein source

(76) Inventors: Brent E. Green, Warren (CA); Martin Schweizer, Winnipeg (CA)

(21) Appl. No.: 13/137,944

(22) Filed: Sep. 22, 2011

Related U.S. Application Data
(60) Provisional application No. 61/344,728, filed on Sep. 22, 2010.

(51) Int. Cl. C07K 1/14

(52) U.S. Cl. 530/378

(57) ABSTRACT

A soy protein product, which may be an isolate, useful for the fortification of soft drinks and sports drinks without precipitation of protein, is prepared by extraction of a soy protein source using aqueous calcium salt solution in a counter-current operation in which the extracting aqueous calcium salt solution flows in counter-current direction to the flow of soy protein source, to form an aqueous soy protein solution, entrained fine particulates are at least partially removed from the aqueous soy protein solution and the pH of the resulting soy protein solution is adjusted to about pH 1.5 to about 4.4 to produce an acidified clear soy protein solution, which may be dried, following optional concentration and diafiltration.

### REFERENCE TO RELATED APPLICATION

This application claims priority under 35 USC 119(e) from U.S. Patent Application No. 61/344,728 filed Sep. 22, 2011.

### FIELD OF THE INVENTION

The present invention is concerned with the provision of oil seed protein products, particularly isolates, by a procedure which involves counter-current extraction of oil seed protein source.

### BACKGROUND OF THE INVENTION

In copending U.S. patent application Ser. Nos. 12/603,087 filed Oct. 21, 2009 (US Patent Publication No. 2010-0098818 published Apr. 22, 2010) and 12/923,897 filed Dec. 12, 2010 (US Patent Publication No. 2011-0038993, published Feb. 17, 2011), assigned to the assignee hereof and the disclosures of which are incorporated herein by reference, there is described the provision of a novel soy protein product having a protein content of at least about 60 wt % (N×6.25) on a dry weight basis, preferably a soy protein isolate having a protein content of at least about 90 wt % (N×6.25) d.b. The soy protein product has a unique combination of properties, namely:
completely soluble in aqueous media at acid pH values of less than about 4.4
heat stable in aqueous media at acid pH values of less than about 4.4
does not require stabilizers or other additives to maintain the protein product in solution
is low in phytic acid
requires no enzymes in the production thereof.

In addition, the soy protein product has no beany flavour or off odours characteristic of soy protein products.

This novel soy protein product is prepared by a method which comprises:
(a) extracting a soy protein source with an aqueous calcium chloride solution to cause solubilization of soy protein from the protein source and to form an aqueous soy protein solution,
(b) at least partially separating the aqueous soy protein solution from residual soy protein source,
(c) optionally diluting the aqueous soy protein solution,
(d) adjusting the pH of the aqueous soy protein solution to a pH of about 1.5 to about 4.4, preferably about 2 to about 4, to produce an acidified clear soy protein solution,
(e) optionally polishing the acidified clear solution to remove residual particulars,
(f) concentrating the aqueous clear soy protein solution while maintaining the ionic strength substantially constant by using a selective membrane technique,
(g) optionally diafiltering the concentrated soy protein solution, and
(h) optionally drying the concentrated soy protein solution.

### SUMMARY OF THE INVENTION

It has been found that, in effecting the extraction of the soy protein source, such as with an aqueous calcium chloride solution in the above process, to cause solubilization of soy protein from the soy protein source, benefits arise in effecting this operation in counter-current manner, with the extracting calcium chloride solution flowing in counter-current direction to the flow of the soy protein source.

The flow of fresh extraction solution against the flow of the soy protein source makes for a more efficient extraction of protein than is seen in batch extraction processes. The counter-current flow extraction process of the invention is not limited to the extraction of soy protein from soy protein source using aqueous calcium chloride solution, but rather may be utilized for the extraction of protein from any oil seed protein source using any counter-current extraction solution, such as water, aqueous calcium chloride or aqueous sodium chloride to achieve the same benefits.

The invention is described herein more particularly with respect to the production of soy protein products. It will be apparent, as described above, that equivalent procedures may be used to form other oil seed protein products.

Accordingly, in one aspect of the present invention, there is provided a method of preparation of a soy protein solution, which comprises:
(a) extracting a soy protein source with an aqueous calcium salt solution by a counter-current method to cause solubilization of soy protein from the soy protein source and to form an aqueous soy protein solution and residual soy protein source,
(b) at least partially separating from the aqueous soy protein solution suspended solids in the aqueous protein solution to provide a clarified aqueous soy protein solution, and
(c) optionally diluting the clarified aqueous soy protein solution,
(d) adjusting the pH of the optionally diluted clarified aqueous soy protein solution to a pH of about 1.5 to about 4.4 to produce an acidified soy protein solution.

The soy protein product produced according to the process herein lacks the characteristic beany flavour of soy protein products and is suitable, not only for protein fortification of acid media, but may be used in a wide variety of conventional applications of protein products, including but not limited to protein fortification of processed foods and beverages, emulsification of oils, as a body former in baked goods and foaming agent in products which entrap gases. In addition, the soy protein product may be formed into protein fibers, useful in meat analogs and may be used as an egg white substitute or extender in food products where egg white is used as a binder. The soy protein product may be used in nutritional supplements. The soy protein product may also be used in dairy analog products or products that are dairy/soy blends. Other uses of the soy protein product are in pet foods, animal feed and in industrial and cosmetic applications and in personal care products.

### BRIEF DESCRIPTION OF DRAWING

FIG. 1 is schematic representation of a batch counter-current extraction process carried out on a soy protein source in accordance with one embodiment of the invention.

### GENERAL DESCRIPTION OF THE INVENTION

Protein solubilization from the soy protein source, which may be soybeans or any soy product or by-product derived from the processing of soybeans, preferably soymeal or flakes, is effected most conveniently using calcium chloride solution, although solutions of other calcium salts may be used. In addition, other alkaline earth metal compounds may be used, such as magnesium salts. Further, extraction of the soy protein from the soy protein source may be effected using calcium salt solution in combination with another salt solution, such as sodium chloride. Additionally, extraction of the soy protein from the soy protein source may be effected using water or other salt solution, such as sodium chloride, with calcium salt subsequently being added to the aqueous soy protein solution produced in the extraction step. Precipitate formed upon addition of the calcium salt is removed prior to subsequent processing.

As the concentration of the calcium salt solution increases, the degree of solubilization of protein from the soy protein source initially increases until a maximum value is achieved. Any subsequent increase in salt concentration does not increase the total protein solubilized. The concentration of calcium salt solution which causes maximum protein solubilization varies depending on the salt concerned. It is usually preferred to utilize a concentration value less than about 1.0 M, and more preferably a value of about 0.10 to about 0.15 M.

In accordance with the present invention, the extraction of the soy protein source is effected in a counter-current manner. Any suitable equipment may be used to effect such operation. In one such apparatus, the soy protein source is continuously conveyed in one direction by a system of driven paddles while the extraction solvent enters the opposite end of the extractor and flows in the opposite direction through the soy protein source, thereby ensuring that the soy protein source is continually extracted with fresher solvent as it moves through the extractor. Paddles move the soy protein source up inclined plates that separate each stage of the counter-current extractor and then drop the soy protein source into the next stage. The only mixing involved takes place when the soy protein source is moved along by the paddles and then as it falls into each successive stage. The paddles remove the residual soy protein source from the final stage and carry it out of the extractor.

The gentle nature of this extraction procedure results in less break-up of the soy protein source than is the case when a batch mixer and decanter are employed. Less breakage of the soy protein source results in lesser numbers of fine particulates in the extract, making it easier to achieve clarity downstream from the extraction step. Also, less break-up of the soy protein source facilitates, if desired, the use of a press to remove retained liquid from the residual soy protein source. Use of a press allows a lower moisture content in the residual soy protein source than the use of certain models of decanters. Lowering the moisture content of residual soy protein source destined for drying as much as possible prior to the drying step is advantageous in that drying costs are reduced.

In another embodiment of the present invention, the soy protein source may be extracted in a counter current type sequence by using a series of stirred tanks with the soy protein source material separated from the extract solution between tanks. Utilizing stirred tanks provides better mixing than the type of extractor described above and so allows more efficient extraction. The soy protein source moves through the system in the opposite direction of the extraction solvent. Each stirred tank can be regarded as one stage of the counter current process. The extracted soy protein source material from a given stage enters the next stirred tank along with the lower protein content extract of the subsequent stage. The extract solution from a given stage is moved to the previous stage to be mixed with higher protein content (less extracted) soy protein source material. The extracted soy protein source material may be separated from the extract solution, in any convenient manner, such as by employing a cross flow sieve or decanter centrifuge. The use of a cross flow sieve is preferred since it reduces the amount of foam produced during the process and minimizes break up of the soy protein source material.

In the counter-current process, the salt solubilization of the protein is effected at a temperature of from about 1° C. to about 100° C., preferably from about 15° C. to about 65° C., more preferably from about 50° C. to about 60° C., for about 1 to about 60 minutes. It is preferred to effect the solubilization to extract substantially as much protein from the soy protein source as is practicable, so as to provide an overall high product yield.

The extraction of the soy protein source is generally conducted at a pH of about 5 to about 11, preferably about 5 to about 7. The pH of the extraction system (soy protein source and calcium salt solution) may be adjusted to any desired value within the range of about 5 to about 11 for use in the extraction step by the use of any convenient food grade acid, usually hydrochloric acid or phosphoric acid, or food grade alkali, usually sodium hydroxide, as required.

The concentration of soy protein source in the calcium salt solution during the solubilization step in the counter-current flow may vary widely. Typical concentration values are about 5 to about 15% w/v.

The protein extraction step with the aqueous salt solution has the additional effect of solubilizing fats which may be present in the soy protein source, which then results in the fats being present in the aqueous phase.

The protein solution resulting from the counter-current extraction step generally has a protein concentration of about 5 to about 50 g/L, preferably about 20 to about 50 g/L.

The aqueous calcium salt solution may contain an antioxidant. The antioxidant may be any convenient antioxidant, such as sodium sulfite or ascorbic acid. The quantity of antioxidant employed may vary from about 0.01 to about 1 wt % of the solution, preferably about 0.05 wt %. The antioxidant serves to inhibit oxidation of any phenolics in the protein solution.

The aqueous soy protein solution resulting from the counter-current extraction step contains small amounts of fines of the solid component. These suspended solids may be separated from the protein solution, in any convenient manner, such as by employing disc centrifugation and/or filtration. The separated solids may be added to the residual soy protein source, may be dried for disposal or may be processed to recover some residual protein. The separated solids may be re-extracted with fresh calcium salt solution and the protein solution yielded upon clarification combined with the initial protein solution for further processing as described below. Alternatively, the separated solids may be processed by a conventional isoelectric precipitation procedure or any other convenient procedure to recover residual protein.

The residual soy protein source may be dried for disposal or processed to recover some residual protein. The residual soy protein source may be processed by a conventional isoelectric precipitation procedure or any other convenient procedure to recover residual protein.

Where the soy protein source contains significant quantities of fat, as described in U.S. Pat. Nos. 5,844,086 and 6,005,076, assigned to the assignee hereof and the disclosures of which are incorporated herein by reference, then the defatting steps described therein may be effected on the aqueous protein solution. Alternatively, defatting of the aqueous protein solution may be achieved by any other convenient procedure.

The aqueous soy protein solution may be treated with an adsorbent, such as powdered activated carbon or granulated activated carbon, to remove colour and/or odour compounds. Such adsorbent treatment may be carried out under any convenient conditions, generally at the ambient temperature of the aqueous protein solution. For powdered activated carbon, an amount of about 0.025% to about 5% w/v, preferably about 0.05% to about 2% w/v, is employed. The adsorbing agent may be removed from the soy solution by any convenient means, such as by filtration.

The resulting aqueous soy protein solution is diluted, generally with about 0.5 to about 10 volumes, preferably about 0.5 to about 2 volumes of aqueous diluent, in order to decrease the conductivity of the aqueous soy protein solution to a value of generally below about 90 mS, preferably about 4 to about 18 mS. Such dilution is usually effected using water, although dilute salt solution, such as sodium chloride or calcium chloride, having a conductivity of up to about 3 mS, may be used.

The diluent with which the soy protein solution is mixed generally has the same temperature as the soy protein solution, but the diluent may have a temperature of about 1° to about 100° C., preferably about 15° to about 65° C., more preferably about 50° to about 60° C.

The diluted soy protein solution then is adjusted in pH to a value of about 1.5 to about 4.4, preferably about 2 to about 4, by the addition of any suitable food grade acid, such as hydrochloric acid or phosphoric acid, to result in a clear aqueous soy protein solution. The clear acidified aqueous soy protein solution has a conductivity of generally below about 95 mS, preferably about 4 to about 23 mS.

The clear acidified aqueous soy protein solution may be subjected to a heat treatment to inactivate heat labile anti-nutritional factors, such as trypsin inhibitors, present in such solution as a result of extraction from the soy protein source material during the extraction step. Such a heating step also provides the additional benefits of reducing the microbial load and improving the clarity of the solution. Generally, the protein solution is heated to a temperature of about 70° to about 160° C. for about 10 seconds to about 60 minutes, preferably about 80° to about 120° C. for about 10 seconds to about 5 minutes, more preferably about 85° to about 95° C. for about 30 seconds to about 5 minutes. The heat treated acidified soy protein solution then may be cooled for further processing as described below, to a temperature of about 2° to about 65° C., preferably about 50° C. to about 60° C.

The clear acidified and optionally heat treated soy protein solution may optionally be polished by any convenient means, such as by filtering, to remove any residual particles from the solution.

The resulting clear acidified aqueous soy protein solution may be directly dried to produce a soy protein product. In order to provide a soy protein product having a decreased impurities content and a reduced salt content, such as a soy protein isolate, the clear acidified aqueous soy protein solution may be processed prior to drying.

The clear acidified aqueous soy protein solution may be concentrated to increase the protein concentration thereof while maintaining the ionic strength thereof substantially constant. Such concentration generally is effected to provide a concentrated soy protein solution having a protein concentration of about 50 to about 300 g/L, preferably about 100 to about 200 g/L.

The concentration step may be effected in any convenient manner consistent with batch or continuous operation, such as by employing any convenient selective membrane technique, such as ultrafiltration or diafiltration, using membranes, such as hollow-fibre membranes or spiral-wound membranes, with a suitable molecular weight cut-off, such as about 3,000 to about 1,000,000 Daltons, preferably about 5,000 to about 100,000 Daltons, having regard to differing membrane materials and configurations, and, for continuous operation, dimensioned to permit the desired degree of concentration as the aqueous protein solution passes through the membranes.

As is well known, ultrafiltration and similar selective membrane techniques permit low molecular weight species to pass therethrough while preventing higher molecular weight species from so doing. The low molecular weight species include not only the ionic species of the food grade salt but also low molecular weight materials extracted from the source material, such as carbohydrates, pigments, low molecular weight proteins and anti-nutritional factors, such as trypsin inhibitors, which are themselves low molecular weight proteins. The molecular weight cut-off of the membrane is usually chosen to ensure retention of a significant proportion of the protein in the solution, while permitting contaminants to pass through having regard to the different membrane materials and configurations.

The concentrated soy protein solution then may be subjected to a diafiltration step using water or a dilute saline solution. The diafiltration solution may be at its natural pH or at a pH equal to that of the protein solution being diafiltered or at any pH value in between. Such diafiltration may be effected using from about 1 to about 40 volumes of diafiltration solution, preferably about 2 to about 25 volumes of diafiltration solution. In the diafiltration operation, further quantities of contaminants are removed from the clear aqueous soy protein solution by passage through the membrane with the permeate. This purifies the clear aqueous protein solution and may also reduce its viscosity. The diafiltration operation may be effected until no significant further quantities of contaminants or visible colour are present in the permeate or until the retentate has been sufficiently purified so as, when dried, to provide a soy protein isolate with a protein content of at least about 90 wt % (N×6.25) d.b. Such diafiltration may be effected using the same membrane as for the concentration step. However, if desired, the diafiltration step may be effected using a separate membrane with a different molecular weight cut-off, such as a membrane having a molecular weight cut-off in the range of about 3,000 to about 1,000,000 Daltons, preferably about 5,000 to about 100,000 Daltons, having regard to different membrane materials and configuration.

Alternatively, the diafiltration step may be applied to the clear acidified aqueous protein solution prior to concentration or to the partially concentrated clear acidified aqueous protein solution. Diafiltration may also be applied at multiple points during the concentration process. When diafiltration is applied prior to concentration or to the partially concentrated solution, the resulting diafiltered solution may then be additionally concentrated. The viscosity reduction achieved by diafiltering multiple times as the protein solution is concentrated may allow a higher final, fully concentrated protein concentration to be achieved. This reduces the volume of material to be dried.

The concentration step and the diafiltration step may be effected herein in such a manner that the soy protein product subsequently recovered contains less than about 90 wt % protein (N×6.25) d.b., such as at least about 60 wt % protein (N×6.25) d.b. By partially concentrating and/or partially diafiltering the clear aqueous soy protein solution, it is possible to only partially remove contaminants. This protein solution may then be dried to provide a soy protein product with lower levels of purity. The soy protein product having a protein content of at least about 60 wt % is still able to produce clear protein solutions under acidic conditions.

An antioxidant may be present in the diafiltration medium during at least part of the diafiltration step. The antioxidant may be any convenient antioxidant, such as sodium sulfite or ascorbic acid. The quantity of antioxidant employed in the diafiltration medium depends on the materials employed and may vary from about 0.01 to about 1 wt %, preferably about 0.05 wt %. The antioxidant serves to inhibit the oxidation of any phenolics present in the concentrated soy protein isolate solution.

The concentration step and the optional diafiltration step may be effected at any convenient temperature, generally about 2° to about 65° C., preferably about 50° to about 60° C., and for the period of time to effect the desired degree of concentration and diafiltration. The temperature and other conditions used to some degree depend upon the membrane equipment used to effect the membrane processing, the desired protein concentration of the solution and the efficiency of the removal of contaminants to the permeate.

There are two main trypsin inhibitors in soy, namely the Kunitz inhibitor, which is a heat-labile molecule with a molecular weight of approximately 21,000 Daltons, and the Bowman-Birk inhibitor, a more heat-stable molecule with a molecular weight of about 8,000 Daltons. The level of trypsin inhibitor activity in the final soy protein product can be controlled by manipulation of various process variables.

As noted above, heat treatment of the clear acidified aqueous soy protein solution may be used to inactivate heat-labile trypsin inhibitors. The partially concentrated or fully concentrated acidified soy protein solution may also be heat treated to inactivate heat labile trypsin inhibitors. When the heat treatment is applied to the partially concentrated acidified soy protein solution, the resulting heat treated solution may then be additionally concentrated.

In addition, the concentration and/or diafiltration steps may be operated in a manner favorable for removal of trypsin inhibitors in the permeate along with the other contaminants. Removal of the trypsin inhibitors is promoted by using a membrane of larger pore size, such as about 30,000 to about 1,000,000 Daltons, operating the membrane at elevated temperatures such as about 30 to about 65° C., preferably about 50° to about 60° C. and employing greater volumes of diafiltration medium, such as about 10 to about 40 volumes.

Acidifying and membrane processing the diluted protein solution at a lower pH of about 1.5 to about 3 may reduce the trypsin inhibitor activity relative to processing the solution at a higher pH of about 3 to about 4.4. When the protein solution is concentrated and diafiltered at the low end of the pH range, it may be desired to raise the pH of the retentate prior to drying. The pH of the concentrated and diafiltered protein solution may be raised to the desired value, for example pH 3, by the addition of any convenient food grade alkali such as sodium hydroxide.

Further, a reduction in trypsin inhibitor activity may be achieved by exposing soy materials to reducing agents that disrupt or rearrange the disulfide bonds of the inhibitors. Suitable reducing agents include sodium sulfite, cysteine and N-acetylcysteine.

The addition of such reducing agents may be effected at various stages of the overall process. The reducing agent may be added with the soy protein source material in the extraction step, may be added to the clarified aqueous soy protein solution following the removal of suspended solids, may be added to the concentrated protein solution before or after diafiltration or may be dry blended with the dried soy protein product. The addition of the reducing agent may be combined with a heat treatment step and membrane processing steps, as described above.

If it is desired to retain active trypsin inhibitors in the concentrated protein solution, this can be achieved by eliminating or reducing the intensity of the heat treatment step, not utilizing reducing agents, operating the concentration and diafiltration steps at the higher end of the pH range, such as pH of about 3 to about 4.4, utilizing a concentration and diafiltration membrane with a smaller pore size, operating the membrane at lower temperatures and employing fewer volumes of diafiltration medium.

The concentrated and optionally diafiltered protein solution may be subject to a further defatting operation, if required, as described in U.S. Pat. Nos. 5,844,086 and 6,005,076. Alternatively, defatting of the concentrated and optionally diafiltered protein solution may be achieved by any other convenient procedure.

The concentrated and optionally diafiltered clear aqueous protein solution may be treated with an adsorbent, such as powdered activated carbon or granulated activated carbon, to remove colour and/or odour compounds. Such adsorbent treatment may be carried out under any convenient conditions, generally at the ambient temperature of the concentrated protein solution. For powdered activated carbon, an amount of about 0.025% to about 5% w/v, preferably about 0.05% to about 2% w/v, is employed. The adsorbent may be removed from the soy protein solution by any convenient means, such as by filtration.

The concentrated and optionally diafiltered clear aqueous soy protein solution may be dried by any convenient technique, such as spray drying or freeze drying. A pasteurization step may be effected on the soy protein solution prior to drying. Such pasteurization may be effected under any desired pasteurization conditions. Generally, the concentrated and optionally diafiltered soy protein solution is heated to a temperature of about 55° to about 70° C., preferably about 60° to about 65° C., for about 30 seconds to about 60 minutes, preferably about 10 minutes to about 15 minutes. The pasteurized concentrated soy protein solution then may be cooled for drying, preferably to a temperature of about 25° to about 40° C.

The dry soy protein product has a protein content of at least about 60 wt % (N×6.25) d.b. Preferably the dry protein product is an isolate with a high protein content, in excess of about 90 wt %, preferably at least about 100 wt %, (N×6.25) d.b.

The soy protein product produced herein is soluble in an acidic aqueous environment, making the product ideal for incorporation into beverages, both carbonated and uncarbonated, to provide protein fortification thereto. Such beverages have a wide range of acidic pH values, ranging from about 2.5 to about 5. The soy protein product provided herein may be added to such beverages in any convenient quantity to provide protein fortification to such beverages, for example, at least about 5 g of the soy protein per serving. The added soy protein product dissolves in the beverage and does not impair the clarity of the beverage, even after thermal processing. The soy protein product may be blended with dried beverage prior to reconstitution of the beverage by dissolution in water. In some cases, modification to the normal formulation of the beverages to tolerate the inclusion of the soy protein product may be necessary where components present in the beverage may adversely affect the ability of the soy protein product to remain dissolved in the beverage.

### EXAMPLES

### Example 1

This Example illustrates a counter-current extraction of defatted soy white flakes in accordance with one embodiment of the invention.

A 3-stage Crown Model IV counter-current extractor (www.crowniron.com) was used in initial testing with soy white flakes. The white flakes used had a protein content of 49.1 wt %. 0.15M calcium chloride solution was used as the extraction solvent. White flakes were fed at a rate of 25 g per minute to the extractor while the solvent was fed at a rate of 180 to 190 ml/minute, equating to an extraction ratio of approximately 13.5% w/v. The rate of extract exiting the extractor was measured at 150 to 160 ml/min., the difference between the feed rate and extract outlet rate being attributed to solvent volume loss due to absorption by the white flakes exiting the extractor.

The initial residence time was set at one hour by adjusting the speed of the chain driven paddles so that the flakes took one hour to pass through the entire extractor. After running for approximately 90 minutes at these settings, residual flakes and extract samples were collected and the residence time was reduced to 45 minutes by increasing the paddle speed and thereby moving the flake through the extractor at a faster rate. Flakes and solvent feed rates were unchanged in order to maintain a 13.5% w/v extraction ratio.

The extractor was run for another hour to again achieve steady state. Samples were then taken and the paddle speed was increased further to target a 30 minute residence time. Once again, the system was run for an hour to achieve steady state before extract and residual flake samples were collected.

Once the proper feed rates were determined, the extractor ran well with no backing up of flakes and very little carry over of solids into the protein extract. This resulted in a solution that was quite clear with very little foam.

The data in Table 1 below compares the clarity (A600) and protein contents (Leco combustion analysis) of the extracts produced by the counter-current extractor to the values obtained for a batch extraction in which residual soy protein source was removed from solution through the use of a decanter. In the batch extraction BW-S015-A14-10A, 30 kg of soy white flake was added to 300 L of 0.15M calcium chloride solution in a tank equipped with overhead mixers. After 30 minutes of mixing, the extract slurry was pumped through a decanter to remove the residual flakes and 251 L of extract solution was recovered having a protein content of 2.52 wt %.

TABLE 1

| | A600 | % protein | Extractability |
|---|---|---|---|
| 1 hour residence time | 0.47 | 2.45% | 31% |
| 45 minutes residence time | 0.95 | 2.46% | 31% |
| 30 minutes residence time | 1.14 | 3.085 | 39% |
| Batch extraction and Decanter | 2.65 | 2.52% | 43% |
| method | | | |

As can be seen in Table 1, the counter-current extractor produced extracts of very similar protein content to the batch extraction but with greatly reduced haze. Although the counter-current extracts resulted in similar protein contents, a higher flake to solvent ratio was used compared to the batch extraction and this could explain the lower yields seen for the counter current trials. However, the significantly lower haze and lack of foam make the counter current extract much easier to process further. Additionally, it was theorized that one hour run time may not be enough to reach steady state, since the entire extractor was filled with calcium chloride solution at the beginning, which will cause a dilution effect.

The protein content in the counter-current extraction increased throughout the test period even though the residence time decreased. This suggests that the protein content was building throughout the run and may not have peaked and reached steady state yet. With this in mind, it should be possible to achieve higher yields if the system were run for longer periods.

### Example 2

This Example illustrates counter-current extraction of defatted soy white flakes in accordance with another embodiment of the invention.

A 5-stage Crown Model IV extractor was used for the experiments of this Example. The additional extraction stages were used in the expectation that they would result in higher extractability due to the additional mixing taking place in the extra stages.

White flakes feed rate was maintained at 131 g per minute while solvent feed rate was 1.13 L per minute. This equates to an extraction ratio of approximately 11.5%. The protein content of the white flakes was 48.7 wt %. The extract outlet rate was approximately 0.9 ml/minute.

The results obtained are set forth in the following Table 2.

TABLE 2

| | Moisture (%) | A600 | % protein |
|---|---|---|---|
| Extract at 10:15 | 95.88 | | 1.39 |
| Extract at 10:45 | 95.35 | | 1.74 |
| Extract at 11:15 | 94.99 | | 1.96 |
| Extract at 11:45 | 94.49 | | 2.24 |
| Extract at 12:15 | 94.28 | | 2.39 |
| Extract at 12:45 | 93.92 | | 2.40 |
| Composite of first 161 L of extract | 96.45 | 0.099 | 1.25 |
| Composite of second 76 L of extract | 94.46 | 0.134 | 2.28 |
| Composite of final 151 L of extract | 94.79 | 0.07 | 2.30 |

As can be seen from the results in Table 2, the protein content in the extract appears to have increased as the extractor ran longer. This can be seen in the 10:15 to 12:45 liquid samples in Table 2, once again suggesting that it takes longer than originally expected to actually achieve steady state in the system.

The A600 values for the composite counter current extract solutions in this trial were significantly lower than the value obtained for the batch extraction with the decanter used for residual white flake removal in Example 1. The higher degree of clarity is desirable as it allows for easier downstream processing.

A protein mass balance for this run is given in Table 3:

TABLE 3

| | Volume (L) | | Protein | Protein | |
|---|---|---|---|---|---|
| | or mass (kg) | | (%) | (kg) | |
| | Protein material IN | | | | |
| White flakes | 31.13 | kg | 48.7 | 15.16 | |
| | | | Total | 15.16 | 100% |
| | Protein in extract OUT | | | | |
| Composite of first 161 L | 161 | L | 1.25 | 2.01 | |
| of extract | | | | | |
| Composite of second 76 L | 76 | L | 2.28 | 1.73 | |
| of extract | | | | | |
| Composite of final 151 L | 151.4 | L | 2.30 | 3.48 | |
| of extract | | | | | |
| | | | Total: | 7.22 | 48% |

As can been seen in Table 3, the overall yield of extracted protein for this run was 48% of the starting protein. This is an improved yield as compared to batch extraction.

### Example 3

The Example illustrates counter-current extraction using multiple extraction tanks in accordance with one embodiment of the invention.

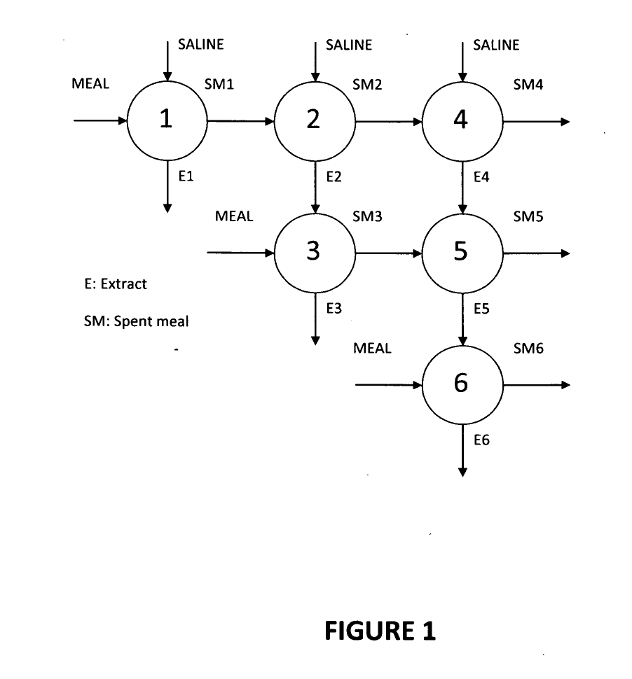

A series of extractions were carried out using defatted soy white flakes to examine counter-current type extraction using a series of batch extraction tanks. The removal of residual white flakes was carried out by the use of screens so as not to excessively break up the flakes. The set of extractions was set up in such a way that the most depleted white flakes were extracted with the freshest solvent while the freshest white flakes were first extracted with saline containing the highest percentage of protein. The method used closely mimics what would happen in a true counter-current extraction but with a much higher degree of mixing. The batch designation for this series of extractions was BW-S017-C31-10A. A schematic of the extraction series is shown in FIG. 1.

An initial 10% w/v extraction in 500 L of 0.15M CaCl₂ was carried out in a tank with overhead mixing. The soy white flakes used had a protein content of 48.7 wt %. Extraction time was 30 minutes. After the 30 minute extraction, the residual or extracted white flakes were removed from solution by pumping the extract slurry through a Kason vibratory sieve housing 16 and 50 mesh screens. The extract solution (E1) was sampled and discarded while the residual white flakes were collected and labelled as SM1.

SM1 was then added to enough fresh 0.15M CaCl₂ to make a 25% w/v extraction and mixed for 15 minutes. The Kason vibratory screen was again used to remove the residual white flakes (SM2) and produce a second extract solution (E2). SM2 was set aside for later use.

Extract E2 was used to extract fresh white flakes. A 10% w/v extraction was carried out with 30 minute mixing. The residual white flakes were removed by the vibratory screen again and labelled SM3. The extract (E3) was sampled and discarded.

SM2 was extracted in enough fresh 0.15M CaCl₂ to make a 25% w/v extraction. An extraction time of 15 minutes was used. The residual white flakes were collected and labelled SM4. The resulting extract was collected and labelled E4.

Extract E4 was used for a 25% extraction of SM3. Extraction time was 15 minutes and the residual white flakes (SM5) were recovered with the vibratory screen again. Extract E5 was collected for use in the final extraction.

Extract E5 was used to do a 10% w/v extraction on fresh white flakes. Extraction time was 30 minutes and the vibratory screen was used to remove the residual white flakes. This final extract solution E6 was analyzed for protein content and clarity before being processed further.

The results obtained are shown in the following Table 4.

TABLE 4

| | Extract | | |
|---|---|---|---|
| | Protein | Extractability** | A600 |
| Extraction 1 | 2.90 | 41.14% | 29.00 |
| Extraction 2 | 1.03 | NA | |
| Extraction 3 | 3.38 | 54% | 29.86 |
| Extraction 4 | 0.45 | NA | |
| Extraction 5 | 0.97 | NA | |
| Extraction 6 | 3.54 | 57% | 121.5 |

**Due to excessive foaming caused by the vibratory sieve used for white flakes separation, the extractability calculation in Table 4 assumes an 80% volume recovery which is typical of the volume recovered when a decanter is used for white flake/extract separation.

As can be seen from the data in Table 4, this method of extraction was very effective at maximizing the amount of protein recovered from the white flakes. The final extract had very high protein content and an actual extractability of 57%.

Although the use of the sieves was undoubtedly better than repeated passes through a decanter, the final extract still had a significantly higher haze value in this trial compared to what was seen for the composite counter current extracts in Examples 1 and 2. It is likely that in the current trial resulted in more fine particles being carried over from one extraction to the next.

The following conclusions can be drawn from the data presented in Examples 1 to 3.

A counter-current type extractor is capable of providing similar or better extractability than traditional batch extractions of soy white flakes while providing a solution of better clarity. The extracted flakes from this method of extraction also tend to maintain their form better than extracted flakes that have been recovered via a decanter. More intact flakes would perform better in a dewatering press which subsequently would reduce the cost of drying the residual flakes.

Also, high extractability can be achieved via multiple stirred extractions in a counter-current set-up. The better mixing and re-extraction of residual soy protein source by counter-current extraction employing decanters and mixing vessels makes a significant difference in the amount of protein extracted.

### SUMMARY OF THE DISCLOSURE

In summary of this disclosure, the present invention provides a method of extracting protein from oil seed protein source by counter-current extraction. Modifications are possible within the scope of the invention.

What we claim is:

1. A method of preparation of a soy protein solution, which comprises:
(a) extracting a soy protein source with an aqueous calcium salt solution by a counter-current method to cause solubilization of soy protein from the soy protein source and to form an aqueous soy protein solution and residual soy protein source,
(b) at least partially separating from the aqueous soy protein solution residual suspended solids in the aqueous protein solution to provide a clarified aqueous soy protein solution, and
(c) adjusting the pH of the clarified aqueous soy protein solution to a pH of about 1.5 to about 4.4 to produce an acidified soy protein solution.

2. The method of claim 1 wherein said counter-current extraction is effected by continuously flowing said soy protein source material in one direction through a multiple zone extraction tank by the use of inclined plates immersed in aqueous calcium salt solution in each of the extraction zones and paddles which move soy protein source up the inclined plates and drop the soy protein source into the next extraction zone and continuously flowing said aqueous calcium salt solution in the opposite direction through said multiple-zone extraction tank.

3. The method of claim 1 wherein said counter-current extraction is effected in a series of stirred tanks with the soy protein source material separated from the aqueous soy protein solution between tanks wherein the soy protein moves through the series of stirred tanks in the opposite direction from the aqueous calcium salt solution.

4. The method of claim 3 wherein the extracted soy protein source material from one extraction stage enters the next stirred tank along with a lower protein content extract of a subsequent extraction stage and wherein the aqueous calcium salt solution from a given stirred tank is transferred to the preceding extraction stage to be mixed with higher protein content soy protein source material.

5. The process of claim 1 wherein said extraction step is effected using an aqueous calcium chloride solution having a concentration of less than about 1.0 M.

6. The process of claim 5 wherein said aqueous calcium chloride solution has a concentration of about 0.10 to about 0.15 M.

7. The process of claim 1 wherein said extraction step is effected at a temperature of about 1° to about 100° C., preferably about 15° to about 65° C., more preferably about 50° to about 60° C.

8. The process of claim 1 wherein said extraction with aqueous calcium salt solution is conducted at a pH of about 5 to about 11.

9. The process of claim 8 wherein said pH is about 5 to about 7.

10. The process of claim 1 wherein said aqueous soy protein solution has a protein concentration of about 5 to about 50 g/L.

11. The process of claim 10 wherein said protein concentration is about 20 to about 50 g/L.

12. The process of claim 1 wherein said aqueous calcium salt solution contains an antioxidant.

13. The process of claim 1 wherein, following said at least part separation step and prior to said pH adjustment step, said aqueous soy protein solution is treated with an adsorbent to remove colour and/or odour compounds from the aqueous soy protein solution.

14. The process of claim 1 wherein, following said at least part separation step and prior to said pH adjustment step, said aqueous soy protein solution is diluted to a conductivity of less than about 90 mS.

15. The process of claim 14 wherein said aqueous soy protein solution is diluted with about 0.5 to about 10 volumes of aqueous diluent to provide a conductivity of said soy protein solution of about 4 to about 18 mS.

16. The process of claim 14 wherein said aqueous diluent has a temperature of about 1° to about 100° C.

17. The process of claim 16 wherein said temperature is about 15° to about 65° C.

18. The process of claim 17 wherein said temperature is about 50° to about 60° C.

19. The process of claim 1 wherein said acidified soy protein solution has a conductivity of less than about 95 mS.

20. The process of claim 19 wherein said conductivity is about 4 to about 23 mS.

21. The process of claim 1 wherein the pH of said aqueous soy protein solution is adjusted to about pH 2 to about 4.

22. The process of claim 1 wherein the pH-adjusted soy protein solution is subjected to a polishing step.

23. The process of claim 1, wherein said acidified aqueous protein solution is subjected to a heat treatment step to inactivate heat-labile anti-nutritional factors.

24. The process of claim 23 wherein the anti-nutritional factors are heat-labile trypsin inhibitors.

25. The process of claim 23 wherein the heat treatment step also pasteurizes the acidified aqueous protein solution.

26. The process of claim 23, wherein said heat treatment is effected at a temperature of about 70° to about 160° C. for about 10 seconds to about 60 minutes.

27. The process of claim 26 wherein said heat treatment is effected at a temperature of about 80° to about 120° C. for about 10 seconds to about 5 minutes.

28. The process of claim 27 wherein said heat treatment is effected at a temperature of about 85° C. to about 95° C. for about 30 seconds to about 5 minutes.

29. The process of claim 23 wherein the heat treated acidified soy protein solution is cooled to a temperature of about 2° to about 65° C. for further processing.

30. The process of claim 29 wherein the heat treated acidified soy protein solution is cooled to a temperature of about 50° to about 60° C. for further processing.

31. The process of claim 23 wherein the heat treated soy protein solution is subjected to a polishing step.

32. The process of claim 1 wherein said acidified clear soy protein solution is dried to provide a soy protein product having a soy protein content of at least about 60 wt % (N×6.25) d.b.

33. The process of claim 1 wherein said acidified clear soy protein solution is concentrated while maintaining the ionic strength thereof substantially constant to produce a concentrated acidified clear soy protein solution having a protein concentration of about 50 to about 300 g/L and the concentrated acidified clear soy protein solution is optionally diafiltered.

34. The process of claim 33 wherein said concentrated acidified clear soy protein solution has a protein concentration of about 100 to about 200 g/L.

35. The process of claim 33 wherein said concentration step is effected by ultrafiltration using a membrane having a molecular weight cut-off of about 3,000 to about 1,000,000 Daltons.

36. The process of claim 35 wherein said membrane has a molecular weight cut-off of about 5,000 to about 100,000 Daltons.

37. The process of claim 33 wherein a diafiltration step is effected using water, acidified water, dilute saline or acidified dilute saline on the acidified clear soy protein solution before or after partial or complete concentration thereof.

38. The process of claim 37 wherein said diafiltration is effected using about 1 to about 40 volumes of diafiltration solution.

39. The process of claim 38 wherein said diafiltration is effected using about 2 to about 25 volumes of diafiltration solution.

40. The process of claim 37 wherein said diafiltration is effected until no significant further quantities of contaminants or visible colour are present in the permeate.

41. The process of claim 37 wherein said diafiltration is effected until the retentate has been sufficiently purified so as, when dried, to provide a soy protein isolate with a protein content of at least about 90 wt % (N×6.25) d.b.

42. The process of claim 37 wherein said diafiltration is effected using a membrane having a molecular weight cut-off of about 3,000 to about 1,000,000 Daltons.

43. The process of claim 42 wherein said membrane has a molecular weight cut-off of about 5,000 to about 100,000 Daltons.

44. The process of claim 37 wherein an antioxidant is present in the diafiltration medium during at least part of the diafiltration step.

45. The process of claim 33 wherein said concentration step and optional diafiltration step are carried out at a temperature of about 2° to about 65° C.

46. The process of claim 45 wherein said temperature is about 50° to about 60° C.

47. The process of claim 33 wherein the partially concentrated or concentrated and optionally diafiltered acidified clear soy protein solution is subjected to a heat treatment step to inactivate heat-labile anti-nutritional factors, including heat-labile trypsin inhibitors.

48. The process of claim 47 wherein said heat treatment is effected at a temperature of about 70° to about 160° C. for about 10 seconds to about 60 minutes, preferably a temperature of about 80° C. to about 120° C. for about 10 seconds to about 5 minutes, more preferably about 85° C. to about 95° C. for about 30 seconds to about 5 minutes.

49. The process of claim 48 wherein the heat treated soy protein solution is cooled to a temperature of about 2° to about 65° C., preferably about 50° to about 60° C., for further processing.

50. The process of claim 1 wherein said acidified clear soy protein solution is concentrated and/or diafiltered while maintaining the ionic strength thereof substantially constant to produce a concentrated and/or diafiltered acidified clear soy protein solution which, when dried, provides a soy protein product having a protein concentration of at least about 60 wt % (N×6.25) d.b.

51. The process of claim 33 wherein said concentrated and optionally diafiltered acidified clear soy protein solution is treated with an adsorbent to remove colour and/or odour compounds.

52. The process of claim 33 wherein said concentrated and optionally diafiltered acidified clear soy protein solution is pasteurized prior to drying.

53. The process of claim 52 wherein said pasteurization step is effected at a temperature of about 55° to about 70° C. for about 30 seconds to about 60 minutes.

54. The process of claim 53 wherein said pasteurization step is effected at a temperature of about 60° to about 65° C. for about 10 to about 15 minutes.

55. The process of claim 41 wherein said concentrated and diafiltered acidified clear soy protein solution is dried to provide a soy protein isolate having a protein content of at least about 90 wt % (N×6.25) d.b.

56. The process of claim 55 wherein said soy protein isolate has a protein content of at least about 100 wt % (N×6.25) d.b.

57. The process of claim 33 wherein the concentration and/or optional diafiltration step are operated in a manner favourable to the removal of trypsin inhibitors.

58. The process of claim 1 wherein a reducing agent is present during the extraction step to disrupt or rearrange the disulfide bonds of trypsin inhibitors to achieve a reduction in trypsin inhibitor activity.

59. The process of claim 33 wherein a reducing agent is present during the concentration and/or optional diafiltration step to disrupt or rearrange the disulfide bonds of trypsin inhibitors to achieve a reduction in trypsin inhibitor activity.

60. The process of claim 50 wherein a reducing agent is added to the concentrated and optionally diafiltered soy protein solution prior to drying and/or the dried soy protein product to disrupt or rearrange the disulfide bonds of trypsin inhibitors to achieve a reduction in trypsin inhibitor activity.
